(19) United States
(12) Patent Application Publication
(10) Pub. No.: US 2011/0112310 A1
(43) Pub. Date: May 12, 2011

(54) AZIRIDINE SYNTHESIS

(75) Inventors: Pranab K. Patra, Singapore (SG); Jayasree Seayad, Singapore (SG); Yugen Zhang, Singapore (SG); Jackie Y. Ying, Singapore (SG)

(73) Assignee: AGENCY FOR SCIENCE, TECHNOLOGY AND RESEARCH, Singapore (SG)

(21) Appl. No.: 12/865,707

(22) PCT Filed: Jan. 30, 2009
(86) PCT No.: PCT/SG2009/000039

§ 371 Date: Jan. 28, 2011

Related U.S. Application Data
(60) Provisional application No. 61/006,770, filed on Jan. 30, 2008.

(51) Int. Cl. C07D 203/14

(52) U.S. Cl. 548/966; 548/967

(57) ABSTRACT

The invention relates to a process for making an aziridine. wherein an aldehyde, a nitroso compound and a Michael acceptor are reacted in the presence of an N-heterocyclic carbene (NHC) catalyst.

### TECHNICAL FIELD

The present invention relates to a process for making aziridines.

### BACKGROUND OF THE INVENTION

Aziridines are known to readily undergo regioselective ring opening reactions, and are therefore useful in organic synthesis. In particular, they find application in synthesis of complex molecules such as pharmaceuticals and natural products. Additionally, many aziridines are themselves biologically active. There is therefore a need for a simple and versatile process for synthesising aziridines. Existing methods for synthesising these compounds involve nitrenes or SN2 reaction of amines, and require multi-step processes to synthesise the substrates. Existing catalytic methods to make aziridines commonly use organometallic reagents, with attendant disadvantages of metal contamination of the product.

### OBJECT OF THE INVENTION

It is an object of the present invention to at least partially satisfy the above need. It is a further object to at least partially overcome at least one of the above disadvantages with existing methods for synthesising aziridines.

### SUMMARY OF THE INVENTION

In a first aspect of the invention there is provided a process for making an aziridine comprising reacting an aldehyde, a nitroso compound and a Michael acceptor in the presence of an N-heterocyclic carbene (NHC) catalyst.

The following options may be used in conjunction with the first aspect, either individually or in any suitable combination.

The process may be a one-pot process. It may be conducted without isolation of any intermediate species. The process may comprise the steps of:
preparing a reaction mixture comprising the aldehyde, the nitroso compound, the Michael acceptor and a precursor, said precursor being convertible to the NHC; and
converting the precursor in the reaction mixture into the NHC.

The precursor (if used) may be convertible by reaction with a base to the NHC. In this event the step of converting may comprise adding the base to the reaction mixture.

The precursor may be a 1,2,4-triazolium salt. It may be an imidazolium salt. It may be a thiazolium salt. It may be a mixture of any two or more of these. The precursor may be chiral. It may be achiral. It may be

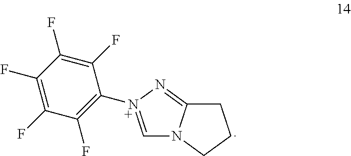

The NHC may be a triazolylidene, e.g. a 1,2,4-triazolylidene. It may be a imidazolylidene. It may be a thiazolylidene. It may be a mixture of any two or more of these. It may be a stable NHC. It may be an unstable NHC. It may be a polymeric NHC. It may be a stable polymeric NHC.

In one form the process comprises the steps of:
preparing a hydroxamic acid by reacting an aldehyde and a nitroso compound in the presence of an N-heterocyclic carbene (NHC) catalyst; and
reacting the hydroxamic acid with a Michael acceptor.

In this form the step of preparing the hydroxamic acid may comprise the steps of:
preparing a reaction mixture comprising the aldehyde, the nitroso compound and a precursor, said precursor being convertible to the NHC; and
converting the precursor in the reaction mixture to the NHC.

In another form the process comprises the steps of:
reacting a nitroso compound with an adduct of an aldehyde and an N-heterocyclic carbene (NHC) to form a hydroxamic acid; and
reacting the hydroxamic acid with a Michael acceptor to produce the aziridine.

The aldehyde may be an aryl aldehyde.

The nitroso compound may be a nitrosoaryl compound.

The Michael acceptor may comprise an olefin group, e.g. a terminal olefin group, having at least one electron withdrawing group attached directly thereto.

In an embodiment there is provided a one-pot process for making an aziridine comprising reacting an aryl aldehyde, a nitrosoaryl compound and a Michael acceptor in the presence of an N-heterocyclic carbene (NHC) catalyst.

In another embodiment there is provided a one-pot process for making an aziridine comprising reacting an aryl aldehyde, a nitrosoaryl compound and a Michael acceptor in the presence of a stable polymeric N-heterocyclic carbene (NHC) catalyst.

In another embodiment there is provided a one-pot process for making an aziridine comprising the steps of:
preparing a reaction mixture comprising an aryl aldehyde, a nitrosoaryl compound, a Michael acceptor and a precursor, said precursor being convertible by reaction with a base to an N-heterocyclic carbene (NHC) catalyst; and
adding the base to the reaction mixture so as to convert the precursor in the reaction mixture into the NHC.

In a second aspect of the invention there is provided a process for making a pharmaceutical product or a natural product or a veterinary product, said process comprising preparing an aziridine according to the process of the first aspect and converting said aziridine to the pharmaceutical product or natural product or veterinary product.

In a third aspect of the invention there is provided the use of an aziridine made by the process of the invention in synthesis. The synthesis may be synthesis of a natural product. It may be synthesis of a pharmaceutical product. It may be synthesis of a veterinary product.

### DETAILED DESCRIPTION OF THE INVENTION

Disclosed herein is a novel NHC-catalyzed synthesis of N-arylaziridines by a three-component reaction of aldehydes, nitrosobenzene and a Michael acceptor. The synthesis, in a preferred embodiment, relies on a catalytic multicomponent synthesis, where an aldehyde, nitrosobenzene and a Michael acceptor in the presence of an organic catalyst such as carbene provides aziridines in a one-pot system.

In this embodiment it is thought that a hydroxamic acid is formed in situ catalytically, and then reacts with the Michael acceptor to provide the aziridine as the final product. This provides a short and efficient synthetic process for generating an aziridine.

In the process of the invention, an aldehyde, a nitroso compound and a Michael acceptor are reacted in the presence of an N-heterocyclic carbene (NHC) catalyst. In a preferred embodiment, the process is conducted as a one-pot process, i.e. there is no isolation of intermediate species.

Many NHCs have limited stability. It may therefore be convenient to generate the NHC in situ. Thus the reagents (aldehyde, nitroso compound and Michael acceptor) may be combined with a precursor to the NHC to form a reaction mixture. The precursor then may be converted in the reaction mixture into the NHC. A convenient precursor for this reaction is a salt corresponding to the NHC. Thus for example a 1,2,4-triazolium combined or an imidazolium compound may be converted to the corresponding carbene by treatment with a base. The precursor may therefore be a catalyst precursor or a precatalyst.

The precursor may be a triazolim salt. It may be a 1,2,4-triazolium salt. The triazolium salt may be a 1-substituted triazolium salt. It may be a 3-substituted triazolium salt. It may be a 4-substituted triazolium salt. It may be a 1,3,4-trisubstituted triazolium salt. It may be a 1,3,4-trisubstituted 1,2,4-triazolium salt. The 1-substituent may be aromatic. It may be electron withdrawing. It may be an electron withdrawing aromatic substituent. It may be an aromatic substituent having one or more electron withdrawing groups, e.g. halogens (F, Cl, Br), trifluoromethyl or other fluorinated (optionally perfluorinated) alkyl etc. It may be a haloaromatic. It may be for example perfluorophenyl or 2,6-dichloro-4-trifluoromethyl. The substituents on the 3 and 4 positions may each, independently, be C1 to C6 straight chain alkyl or C3 to C6 branched alkyl, or they may, together with C3 and N4 of the triazolium ring, form a ring structure having between 4 and 8 atoms. The C1 to C6 straight chain alkyl may be methyl, ethyl, propyl, butyl, pentyl or hexyl. The branched alkyl may be isopropyl, isobutyl, t-butyl, neopentyl or some other C3 to C6 branched alkyl group. One or both of the substituents on the 3 and 4 positions may be chiral. In the event that they, together with C3 and N4 the triazolium ring, form a ring structure having between 4 and 8 atoms, the ring structure may be chiral. It may have a chiral centre in the ring structure. It may have a chiral substituent on the ring structure. The ring structure may have 5 ring atoms. It may have 6 ring atoms. Other than N4 of the triazolium ring, all of the ring atoms may be carbon. Alternatively one or more may be a heteroatom, e.g. N, O or S. The ring structure may be fused with a second ring structure (which may have for example 4, 5, 6, 7 or 8 ring atoms) which may be aliphatic or may be aromatic. The second ring structure may be fused to a third ring structure and so forth. The third ring structure (and, independently any further ring structures) may have for example 4, 5, 6, 7 or 8 ring atoms. It may be aliphatic or may be aromatic. It may be carbocyclic or may have one or more heteroatoms, e.g. N, O or S.

Other suitable precursors include imidazolium salts and thiazolium salts. Any or all of the precursor types described may be monomeric. They may be dimeric. They may be trimeric. They may be oligomeric. They may be polymeric.

The precursor may be achiral. It may be chiral. It may be optically active. It may have a an optical purity of about 20 to about 100%, or about 50 to 100, 80 to 100, 90 to 100, 95 to 100, 20 to 50, 50 to 90, 70 to 90 or 80 to 90%, e.g. about 20, 30, 40, 50, 60, 70, 80, 90, 95, 96, 97, 98, 99, 99.5, 99.9 or 100%. The NHC may be chiral. It may be optically active. It may have a an optical purity of about 20 to about 100%, or about 50 to 100, 80 to 100, 90 to 100, 95 to 100, 20 to 50, 50 to 90, 70 to 90 or 80 to 90%, e.g. about 20, 30, 40, 50, 60, 70, 80, 90, 95, 96, 97, 98, 99, 99.5, 99.9 or 100%.

The counterion of the triazolium salt (or other charged precursor) may be any suitable, preferably unreactive, counterion, e.g. a halide (such as chloride or bromide) or a tetrafluoroborate.

Suitable precursors include:

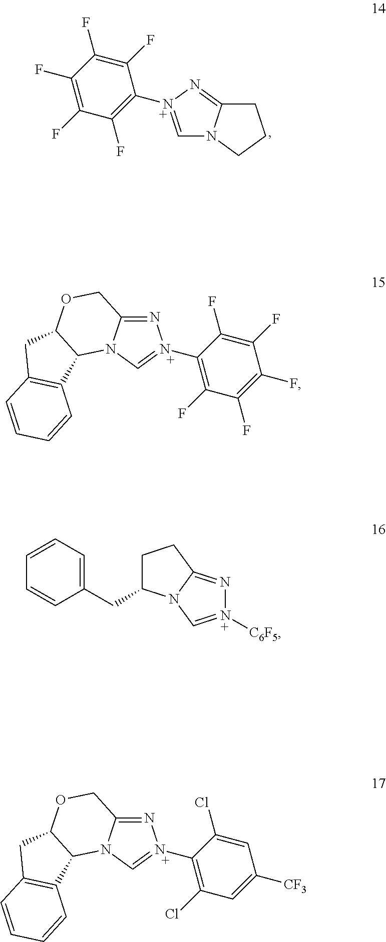

and optical isomers of any of these. Mixtures comprising any two or more of the above may also be used. Other suitable precursors (triazolium salts, imidazolium salts etc.) are provided in WO2008/115153 (N-Heterocyclic carbene (NHC) catalyzed synthesis of hydroxamic acids), the entire contents of which are incorporated herein by cross-reference.

The base used to convert the precursor to the NHC may be a hydride such as sodium hydride. It may be an alkoxide such as potassium t-butoxide. It may be an amine. It may be a tertiary amine. It may be a bridgehead amine. It may be a bicyclic amine. It may for example be 1,8-diazabicyclo[5.4.0] undec-7-ene (DBU).

In the event that the process of the present invention does not comprise the step of preparing the NHC, the NHC may nevertheless have a structure corresponding to (or obtainable from) a precursor as described above.

The NHC may be a triazolylidene (e.g. a 1,2,4-triazolylidene) or an imidazolylidene or a thiazolylidene. It may be monomeric. It may be dimeric. It may be oligomeric. It may be polymeric. It may be a stable NHC. It may be a stable polymeric NHC. It may be a stable polymeric triazolylidene (e.g. 1,2,4-triazolylidene) or a stable polymeric imidazolylidene or a stable polymeric thiazolylidene or a stable copolymeric NHC comprising monomer units of any two or all of triazolylidene, imidazolylidene and thiazolylidene monomer units. It may for example be a polyimidazolylidene. These may be as described in (WO 2007/114793, Polyimidazolium salts and poly-NHC-metal complexes), the entire contents of which are incorporated herein by cross-reference. Examples of such structures include:

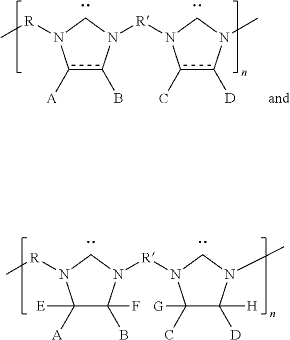

wherein A to H may be, independently, hydrogen, alkyl, aryl, alkenyl, alkynyl or similar, represents either a single or a double bond, R and R′ are linker groups and n is an integer of sufficient size that the structure represents a polymer (e.g. about 10 to about 100, or about 10 to 50, 10 to 20, 20 to 100, 50 to 100 or 20 to 50, e.g. about 10, 15, 20, 25, 30, 35, 40, 45, 50, 60, 70, 80, 90 or 100). The NHC may be soluble. It may be soluble in the reaction mixture. It may be insoluble. It may be insoluble in the reaction mixture. In the event that it is insoluble (which may be the case if it is a polymeric NHC), it may be separated after the reaction has been completed by filtration, centrifugation or other suitable solid separation technique. It may subsequently be regenerated and/or reused in a subsequent reaction. The catalytic activity of the NHC in a subsequent reaction may be at least about 80% of that in the previous reaction, or at least about 85, 90 or 95%. In the event that the NHC is stable, it may be added to the reaction mixture (i.e. the mixture of Michael acceptor, aldehyde and hydroxamic acid) or it may be generated in situ.

The reaction mixture may be metal-free. The process may be metal free. The reaction mixture and/or the process may be free of heavy metals. They may be free of metals other than alkali metals. They may be free of metals other than that introduced with a base used to generate the NHC. They may be free of metals which complex with the NHC.

Whereas a preferred form of the reaction is a one-pot process as described above, the process may be conducted as a two step process. It is thought that the process proceeds by initial reaction of the aldehyde and the nitroso compound to a hydroxamic acid, catalysed by the NHC. The hydroxamic acid is then thought to react in situ with the Michael acceptor to generate the final aziridine product. Thus in one form of the process the intermediate hydroxamic acid may be generated initially, by reacting the aldehyde with the nitroso compound in the presence of the NHC as a catalyst. The hydroxamic acid may then be converted in a separate step to the aziridine by reaction with the Michael acceptor. The second of these steps may be conducted with or without isolation of the hydroxamic acid. Thus the first reaction may be conducted and the Michael acceptor then added directly to the reaction mixture, or the first reaction may be conducted and the hydroxamic acid isolated from the reaction mixture and then combined with the Michael acceptor. In each case, the NHC may be generated from a suitable precursor as described above.

Further, it is thought that the catalytic process by which the NHC catalyses reaction of the aldehyde and the nitroso compound to the hydroxamic acid involves an intermediate adduct of the aldehyde with the nitroso compound. Thus this adduct may be prepared separately and added (commonly in a catalytic amount) to the mixture of the aldehyde, nitroso compound and Michael acceptor (for the one-pot form of the reaction) or to the mixture of aldehyde and nitroso compound (in the two step form). In this case, the adduct would function as the precursor described earlier, but would not require use of base to generate the NHC. As further alternative, the adduct may be prepared separately and added to a mixture of the nitroso compound and the Michael acceptor. In this alternative, the adduct would react with the nitroso compound to form the hydroxamic acid, which could then react in situ with the Michael acceptor. In yet a further alternative, the adduct may be added to the nitroso compound so as to form the hydroxamic acid. In a separate step (with or without separation of the hydroxamic acid), the Michael acceptor may be added so as to form the aziridine. In the latter two alternatives, the adduct would be added in roughly equimolar amounts relative to other reagent(s), as it would be the only source of the aldehyde portion.

The aldehyde may be an aryl aldehyde. It may be a benzaldehyde, optionally substituted. It may be a naphthyl aldehyde or an aldehyde derivative of a polycyclic aromatic hydrocarbon or of a heteroaromatic compound such as pyridine, pyrrole, furan, thiophene. Alternatively the aldehyde may be an alkyl aldehyde, an alkenyl aldehyde (conjugated or unconjugated), an alkynyl aldehyde (conjugated or unconjugated), an arylalky aldehyde (e.g. a phenylacetaldehyde), an arylalkenyl aldehyde, an arylalkynyl aldehyde, or some other aldehyde. In the above groups of aldehydes, the aryl group may be an aromatic hydrocarbon group such as phenyl, napthyl, anthracyl or other polycyclic aromatic hydrocarbyl, or may be heteroaryl, e.g. pyridinyl, furyl, thiopheneyl, pyrrolyl or some other heteroaryl group. It may optionally be substituted. It may be an α-branched aldehyde. It may be an α-aryl aldehyde. It may be an α-aryl α-alkyl aldehyde.

The nitroso compound may be a nitrosoaryl compound. It may be nitrosobenzene. It may be a substituted nitrosobenzene. It may be a nitrosoheteroaromatic (e.g. a nitroso substituted pyridine, pyrrole, furan or thiophene), optionally substituted in addition to the nitroso substituent. It may be a nitroso substituted bicyclic, tricyclic or polycyclic aromatic hydrocarbon, optionally substituted in addition to the nitroso substituent. Alternatively it may be a nitrosoalkyl compound. The alkyl group may be straight chain, branched chain or cyclic or a combination of any two or all of these. The nitroso compound may be optionally substituted in addition to the nitroso group or it may be unsubstituted other than the nitroso group. It may be a nitrosoalkene (conjugated or unconjugated). It may be a nitrosoalkyne (conjugated or unconjugated). It may be a nitrosoalkylarene. It may be a nitrosoarylalkane. It may be a nitrosoalkenylarene. It may be a nitrosoalylalkene. It may be a nitrosoalkynylarene. It may be a nitrosoarylalkyne. It may be some other type of nitroso compound. Any or all of these may be optionally substituted in addition to the nitroso substituent.

The Michael acceptor may be an olefin. It may be an electron deficient olefin. It may be an olefin with an electron withdrawing group. The Michael acceptor may comprise a terminal olefin group having at least one electron withdrawing group attached directly thereto. It may be an acrylate, a methacrylate, an acrylamide, a methacrylamide, an acrylonitrile, a methacrylonitrile or some other suitable Michael acceptor. It may for example be methyl acrylate, methyl methacrylate, dimethyl itaconate, t-butyl acrylate or acrylonitrile. The nature of the Michael acceptor depends on the desired aziridine product, in particular on the desired C2 and C3 substituents of the aziridine. The Michael acceptor may be a non-terminal olefin Suitable examples include maleic anhydride, maleimide, dialkyl (e.g. dimethyl or diethyl) maleate, dialkyl (e.g. dimethyl or diethyl) fumarate etc.

Any one or more of the reagents and the NHC may be chiral. It (they) may be optically active. The aldehyde may be chiral. It may be optically active. The nitrosoalkyl compound may be chiral. It may be optically active. The Michael acceptor may be chiral. It may be optically active. The precursor may be chiral. It may be optically active. The NHC may be chiral. It may be optically active. More than one of these may be chiral and/or optically active. In the event that at least one of these is chiral, the resulting aziridine may be chiral. In the event that at least one of these is optically active, the resulting aziridine may be optically active. Any one of the above may, independently, have an enantiomeric excess, or optical purity, of about 20 to about 100%, or about 50 to 100, 80 to 100, 90 to 100, 95 to 100, 20 to 50, 50 to 90, 70 to 90 or 80 to 90%, e.g. about 20, 30, 40, 50, 60, 70, 80, 90, 95, 96, 97, 98, 99, 99.5, 99.9 or 100%.

The process described herein may be conducted under an inert atmosphere. It may be conducted under nitrogen, argon, helium, carbon dioxide or a mixture of any two or more of these. It may be conducted under some other inert atmosphere. It may be conducted in a solvent. The solvent may be a polar solvent. It may be an aprotic solvent. It may be a halogenated solvent. It may for example be chloroform, dichloromethane, diethyl ether, tetrahydrofuran, 1,4-dioxane, tetrahydropyran, toluene or some other suitable solvent or it may be a mixture of any two or more such suitable solvents. The reaction may be conducted at about 0 to about 50° C., or about 0 to 40, 0 to 30, 0 to 20, 10 to 50, 20 to 50, 30 to 50, 10 to 30 or 15 to 25° C., e.g. about 0, 5, 10, 15, 20, 25, 30, 35, 40, 45 or 50° C. It is commonly conducted at or below the normal boiling point of the solvent, so as to avoid use of a pressure vessel. The time required for the reaction will depend on the nature of the reagents and the NHC (optionally of the precursor) and on the temperature at which the reaction is conducted. It may take from about 1 to about 24 hours, or about 1 to 12, 1 to 6, 1 to 3, 6 to 24, 12 to 24, 18 to 24, 3 to 12, 6 to 12, 3 to 6, 12 to 18, 12 to 15 or 15 to 18 hours, e.g. about 1, 2, 3, 4, 5, 6, 7, 8, 9, 10, 11, 12, 15, 18, 21 or 24 hours. The reaction may be conducted with agitation, optionally continuous agitation. The agitation may comprise stirring, shaking, sonicating, mixing or some other form of agitation, or may comprise more than one of these, either simultaneously or sequentially.

As the aldehyde, nitroso compound and Michael acceptor do not readily react in the absence of the NHC, it is common to mix these reagents (preferably in a solvent) and then add or generate the NHC. In one embodiment of the reaction, the aldehyde, nitroso compound, Michael acceptor and precursor are mixed (preferably in a solvent) to form a reaction mixture. The aldehyde, nitroso compound, Michael acceptor and precursor may each, independently, be in solution in the reaction mixture. They may all be in solution in the reaction mixture. Any two or more may be in solution in the reaction mixture. In particular it is preferred that at least the aldehyde, nitroso compound and Michael acceptor are in solution in the reaction mixture. Addition of a base to the reaction mixture then generates the NHC in situ, leading to rapid reaction of the nitroso compound and the aldehyde catalysed by the NHC to form the hydroxamic acid, which then reacts with the Michael acceptor to form the aziridine.

The molar ratio of aldehyde compound to nitroso compound may be about 0.5 to about 2 (i.e. 1:2 to about 2:1) or about 0.5 to 1, 1 to 2, 0.8 to 1.5, 0.9 to 1.1 or 0.95 to 1.05, e.g. about 0.5, 0.6, 0.7, 0.8, 0.9, 0.95, 1, 1.05, 1.1, 1.2, 1.3, 1.4, 1.5, 1.6, 1.7, 1.8, 1.9 or 2.

The base may be added in molar excess over the precursor. Commonly the molar ratio of precursor to base is about 1 to about 50 (i.e. 1:1 to about 50:1). Stoichiometric base is required to convert the hydroxamic acid intermediate to rearrange and react with Michael acceptor. The molar ratio of precursor to base may be about 5 to 50, 10 to 50, 25 to 50, 1 to 10, 1 to 25, 10 to 25 or 25 to 40, e.g. about 1, 1.1, 1.2, 1.3, 1.4, 1.5, 1.6, 1.7, 1.8, 1.9, 2, 2.5, 3, 4, 5, 6, 7, 8, 9, 10, 15, 20, 25, 30, 35, 40, 45 or 50. The base may be added in approximately molar equivalent amount to the quantity of aldehyde. The ratio of aldehyde to base may be about 0.8 to about 1.2 (i.e. about 0.8:1 to about 1.2:1), or about 0.8 to 1, 1 to 1.2 or 0.9 to 1.1, e.g. about 0.8, 0.85, 0.9, 0.95, 1, 1.05, 01.1, 1.15 or 1.2. It is thought that this quantity of base is required in order to facilitate rearrangement of an intermediate hydroxamic acid so as to react with the Michael acceptor.

The precursor or NHC may be used in a mol % relative to nitroso compound of about 1 to about 10%, or about 1 to 5, 1 to 2, 2 to 10, 5 to 10 or 2 to 5%, e.g. about 1, 1.5, 2, 2.5, 3, 3.5, 4, 4.5, 5, 5.5, 6, 6.5, 7, 7.5, 8, 8.5, 9, 9.5 or 10%. It may be used in a catalytic amount.

The Michael acceptor may be used in a molar excess over the nitroso compound. The molar ratio of Michael acceptor to nitroso compound may be about 1 to about 2 (i.e. about 1:1 to about 2:1) or about 1 to 1.5, 1 to 1.2, 1.2 to 2, 1.5 to 2 or 1.2 to 1.5, e.g. about 1, 1.1, 1.2, 1.3, 1.4, 1.5, 1.6, 1.7, 1.8, 1.9 or 2.

The concentration of the nitroso compound in the solvent may be about 100 to 1000 mM, or about 100 to 500, 100 to 250, 250 to 1000, 500 to 1000 or 250 to 500 mM, e.g. about 100, 150, 200, 250, 300, 350, 400, 450, 500, 550, 600, 650, 700, 750, 800, 850, 900, 950 or 1000 mM.

Following completion of the reaction, the product (i.e. the aziridine) may be isolated from the reaction mixture, i.e. from any unreacted starting materials, byproducts, solvents etc. In a suitable isolation process, the reaction mixture is poured into water and extracted into a suitable organic solvent, e.g. diethyl ether, chloroform, toluene, dichloromethane, ethyl acetate etc. The organic extract may be washed e.g. with water, sodium chloride solution etc. It may be dried over a common dessicant such as an anhydrous salt (calcium chloride, sodium sulphate etc.) or molecular sieve. Further purification may be effected using one or more chromatographic techniques and/or by crystallisation, recrystallisation, sublimation or some other suitable method. Suitable chromatographic techniques include preparative thin layer chromatography, flash column chromatography, preparative hplc, preparative gc or some other form of preparative chromatography.

The aziridines made by the present invention may be used in organic synthesis. They may be used as synthons. They may for example be used in the synthesis of a pharmaceutical product or in the synthesis of a natural product or in the synthesis of a veterinary product. They may be N-substituted aziridines. They may be 2-substituted aziridines. They may be 1,2-disubstituted aziridines. They may be 3-unsubstituted aziridines. They may be 3-substituted aziridines. They may be 1,2,3-trisubstituted aziridines. They may be optically active. They may be chiral. They may be achiral. They may be racemic.

N-heterocyclic carbene (NHC) catalysis has evolved as an efficient method for metal-free carbon-carbon bond formation via the nucleophilic “Breslow intermediate” 2 (Eq. 1) or the homoenolate equivalent species 10 (Eq. 2). Depending on the electrophiles, different types of reactions are possible via both intermediates. Key examples for the former path are benzoin condensation, wherein an aryl aldehyde acts as the electrophile, and Stetter reaction, in which a Michael acceptor takes the role of an electrophile.

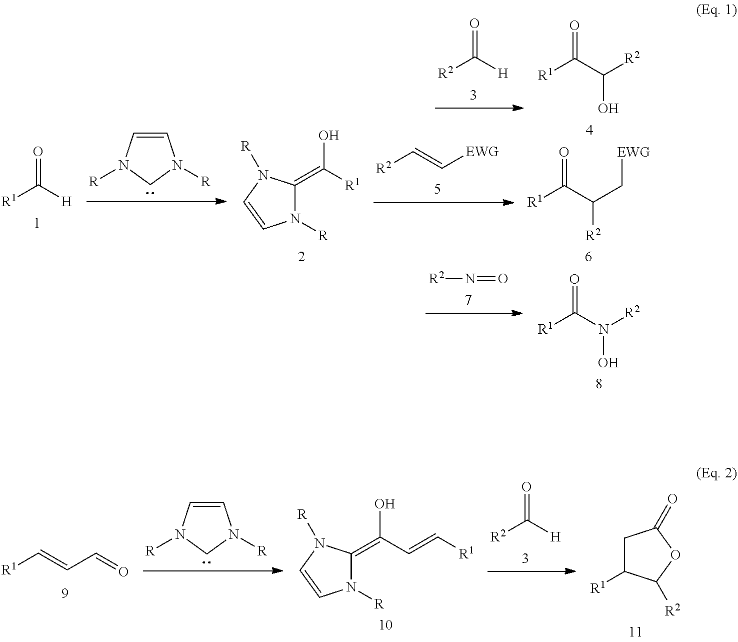

Recently the inventors have developed NHC-catalyzed C—N bond forming reactions using nitroso compounds as electrophiles forming N-arylhydroxamic acids (8) (WO2008/115153) or N-phenylisoxazolidinones (11) and the corresponding β-aminoacid esters.

The present invention relates to a three-component reaction of an aldehyde, a nitroso compound and a Michael acceptor to form the corresponding N-arylaziridines (Scheme 1).

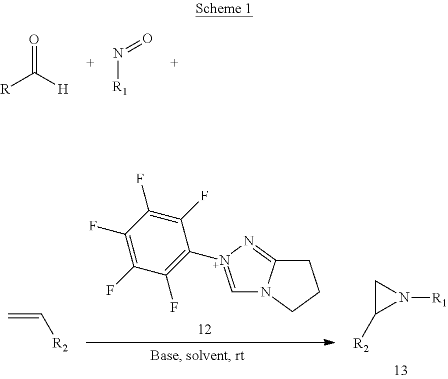

The reaction between hydroxamic acids and acryloyl derivatives forming. N-arylaziridines has been reported previously [(a) Pereira, M. M.; Santos, P. P. O.; Reis, L. V.; Lobo, A. M.; Prabhakar, S. J. Chem. Soc. Chem. Commun. 1993, 38. (b) Aires-de-Sousa, J., Prabhakar, S.; Lobo, A. M.; Rosa, A. M.; Gomes, M. J. S.; Corvo, M. C.; Williams, D. J.; White, A. J. P. Tetrahedron: Asym. 2001, 12, 3349], however these reports failed to demonstrate the convenient three component synthesis from an aldehyde, a nitroso compound and a Michael acceptor disclosed herein.

In the present work it was found that benzaldehyde, nitrosobenzene and methyl acrylate react in the presence of the NHC catalyst generated from the triazolium salt 12 and NaH, forming methyl 1-phenylaziridine-2-carboxylate 13a in excellent yields. The scope of this reaction was extended further by varying the Michael acceptors to synthesize various N-arylaziridine derivatives in high yields (Table 1).

### Examples

Reactions were monitored by thin layer chromatography using 0.25-mm E. Merck silica gel coated glass plates (60F-254) with UV light to visualize the course of reaction. Flash column chromatography was performed using CombiFlash (ISCO, Inc.). Chemical yields referred to pure isolated substances. Gas chromatography-mass spectrometry (GC-MS) was conducted using Shimadzu GC-2010 coupled with GCMS-QP2010. ¹H and ¹³C NMR spectra were obtained using a Brucker AV-400 (400 MHz) spectrometer. Chemical shifts were reported in ppm from tetramethylsilane with the solvent resonance as the internal standard. Data were reported in the following order: chemical shift in ppm (δ) (multiplicity were indicated by br (broadened), s (singlet), d (doublet), t (triplet), q (quartet), m (multiplet)); coupling constants (J, Hz); integration; assignment. All reactions were performed in oven-dried (140° C.) or flame-dried glassware under an inert atmosphere of dry N₂ or argon. All solvents were anhydrous and purchased from Aldrich or Fluka.

### Procedure for the NHC-Catalyzed Three-Component Synthesis of N-Phenylaziridines

NaH (1 mmol) was added under argon to a solution of benzaldehyde (106 mg, 1.0 mmol), nitrosobenzene (107 mg, 1.0 mmol), Michael acceptor (1.3 mmol) and triazolium salt 12 (2.5 mol %) in tetrahydrofuran (THF) (5 mL). The mixture was stirred at room temperature overnight, poured into water (20 mL), and extracted with ether (3×10 mL). The combined ether extracts were washed with brine (20 mL), dried (Na₂SO₄), and concentrated. The pure product was obtained through flash silica gel column chromatography of the residue using hexane and ethyl acetate as the eluents.

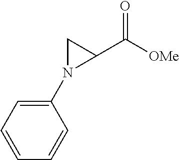

### Methyl 1-Phenylaziridine-2-carboxylate

Yield: 82%. ¹H NMR (400 MHz, CDCl₃): δ 7.28-7.24 (m, 2H, Ar—H), 7.04-7.00 (m, 3H, Ar—H), 3.82 (s, 3H, OMe), 2.81 (dd, J=3.1, 6.3 Hz, 1H, CH₂), 2.68 (dd, J=1.8, 3.1 Hz, 1H, CH), 2.33 (dd, J=1.8, 6.3 Hz, 1H, CH₂). ¹³C NMR (100 MHz, CDCl₃): δ 170.6 (Cq, CO), 152.3 (Cq-Ar), 129.1, 123.4, 120.6 (C—Ar), 52.6 (OCH₃), 37.4 (CH), 33.8 (CH₂). MS (EI): 177 (M⁺), 162, 132, 118, 104, 91, 77.

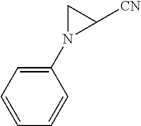

### 1-Phenylaziridine-2-carbonitrile

Yield: 71%. ¹H NMR (400 MHz, CDCl₃): δ 7.34-7.29 (m, 2H, Ar—H), 7.12-7.08 (m, 1H, Ar—H), 7.05-7.02 (m, 2H, Ar—H), 2.79-2.76 (br m, 1H, CH₂), 2.72 (br d, J=2.0 Hz, 1H, CH), 2.49 (br d, J=6.1 Hz, 1H, CH₂). ¹³C NMR (100 MHz, CDCl₃): δ 150.4 (Cq-Ar), 129.4, 124.3, 120.5 (C—Ar), 117.5 (Cq, CN), 33.4 (CH₂), 24.1 (CH). MS (EI): 144 (M⁺), 129, 116, 104, 91, 77.

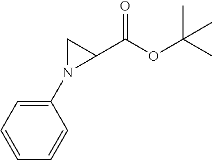

### Tert-butyl 1-phenylaziridine-2-carboxylate

Yield: 87%. ¹H NMR (400 MHz, CDCl₃): δ 7.27-7.23 (m, 2H, Ar—H), 7.03-6.99 (m, 3H, Ar—H), 2.70 (dd, J=3.2, 6.2 Hz, 1H, CH₂), 2.61 (dd, J=1.8, 3.2 Hz, 1H, CH), 2.26 (dd, J=1.8, 6.2 Hz, 1H, CH₂), 1.51 (s, 9H, tBu). ¹³C NMR (100 MHz, CDCl₃): δ 169.1 (Cq, CO), 152.7 (Cq-Ar), 129.0, 123.1, 120.7 (C—Ar), 81.9 (Cq-tBu), 38.4 (CH), 33.5 (CH₂), 28.0 (CH₃). MS (EI): 219 (M⁺), 163, 118, 104, 91, 77.

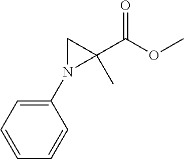

### Methyl 2-methyl-1-phenylaziridine-2-carboxylate

Yield: 67%. ¹H NMR (400 MHz, CDCl₃): δ 7.27-7.22 (m, 2H, Ar—H), 7.03-6.98 (m, 1H, Ar—H), 6.90-6.87 (m, 2H, Ar—H), 3.70 (s, 3H, OMe), 2.85 (dd, J=0.6, 1.3 Hz, 1H, CH₂), 2.19 (d, J=1.3 Hz, 1H, CH₂), 1.34 (s, 3H, CH₃). ¹³C NMR (100 MHz, CDCl₃): δ 171.7 (Cq, CO), 148.5 (Cq-Ar), 128.9, 122.8, 120.7 (C—Ar), 52.5 (OCH₃), 41.4 (Cq), 38.9 (CH₂), 16.0 (CH₃). MS (EI): 191 (M⁺), 176, 132, 118, 104, 91, 77.

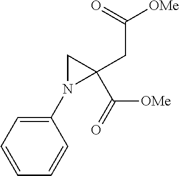

### Methyl 2-((methoxycarbonyl)methyl)-1-phenylaziridine-2-carboxylate

Yield: 69%. ¹H NMR (400 MHz, CDCl₃): δ 7.26-7.22 (m, 2H, Ar—H), 7.02-6.98 (m, 1H, Ar—H), 6.96-6.93 (m, 2H, Ar—H), 3.72, 3.61 (2 s, 6H, 2OMe), 3.03 (d, J=17.2 Hz, 1H, CH₂), 2.95 (dd, J=0.6, 1.0 Hz, 1H, CH₂), 2.45 (d, J=17.2 Hz, 1H, CH₂), 2.41 (d, J=1.0 Hz, 1H, CH₂). ¹³C NMR (100 MHz, CDCl₃): δ 171.0, 169.7 (Cq, CO), 148.5 (Cq-Ar), 129.0, 123.1, 120.3 (C—Ar), 52.5, 52.0 (OCH₃), 42.3 (Cq), 37.7 (CH₂), 37.0 (CH₂). MS (EI): 249 (M⁺), 234, 190, 176, 162, 130, 117, 104, 91, 77.

TABLE 1
Synthesis of N-arylaziridines

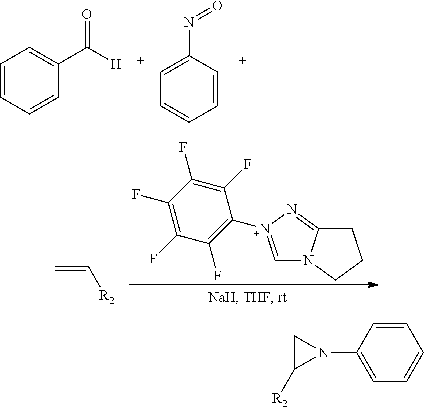

| | | | Iso- |
|---|---|---|---|
| | | | lated |
| En- | | | yield, |
| try | Michael acceptor | Product | % |
| 1 | 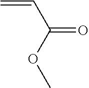 | 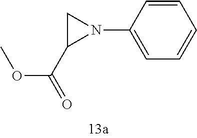 | 82 |
| 2 | 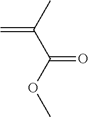 | 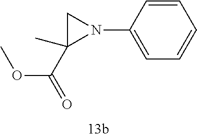 | 67 |
| 3 | 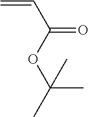 | 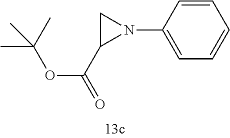 | 87 |
| 4 | 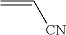 | 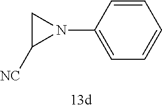 | 71 |
| 5 | 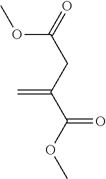 | 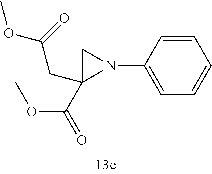 | 69 |

1. A process for making an aziridine comprising reacting an aldehyde, a nitroso compound and a Michael acceptor in the presence of an N-heterocyclic carbene (NHC) catalyst.

2. The process of claim 1, said process being a one-pot process.

3. The process of claim 1 wherein the NHC is a triazolylidene or an imidazolylidene or a thiazolylidene.

4. The process of claim 1 comprising the steps of:
preparing a reaction mixture comprising the aldehyde, the nitroso compound, the Michael acceptor and a precursor, said precursor being convertible to the NHC; and
converting the precursor in the reaction mixture into the NHC.

5. The process of claim 4 wherein the precursor is convertible by reaction with a base to the NHC and the step of converting comprises adding the base to the reaction mixture.

6. The process of claim 5 wherein the precursor is a 1,2,4-triazolium salt or an imidazolium salt or a thiazolium salt.

7. The process of claim 6 wherein the precursor is

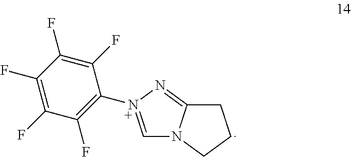

8. The process of claim 1 wherein the process comprises the steps of:
preparing a hydroxamic acid by reacting the aldehyde and the nitroso compound in the presence of the N-heterocyclic carbene (NHC) catalyst; and
reacting the hydroxamic acid with the Michael acceptor.

9. The process of claim 8 wherein the step of preparing the hydroxamic acid comprises the steps of:
preparing a reaction mixture comprising the aldehyde, the nitroso compound and a precursor, said precursor being convertible to the NHC; and
converting the precursor in the reaction mixture to the NHC.

10. The process of claim 9 wherein the precursor is convertible by reaction with a base to the NHC and the step of converting comprises adding the base to the reaction mixture.

11. The process of claim 10 wherein the precursor is a 1,2,4-triazolium salt or an imidazolium salt or a thiazolium salt.

12. The process of claim 11 wherein the precursor is

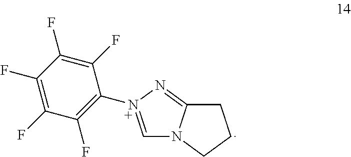

13. The process of claim 1 wherein the process comprises the steps of:
reacting the nitroso compound with an adduct of the aldehyde and the N-heterocyclic carbene (NHC) to form a hydroxamic acid; and
reacting the hydroxamic acid with a Michael acceptor to produce the aziridine.

14. The process of claim 13 wherein the NHC is derived from 1,2,4-triazolium salt or an imidazolium salt or a thiazolium salt.

15. The process of claim 14 wherein the 1,2,4-triazolium salt is

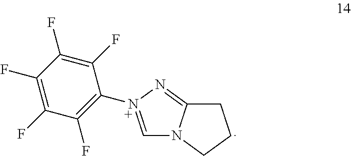

16. The process of claim 1 wherein the aldehyde is an aryl aldehyde.

17. The process of claim 1 wherein the nitroso compound is a nitrosoaryl compound.

18. The process of claim 1 wherein the Michael acceptor comprises a terminal olefin group having at least one electron withdrawing group attached directly thereto.

19. A process for making a pharmaceutical product or natural product, said process comprising preparing an aziridine according to the process of claim 1 and converting said aziridine to the pharmaceutical product or natural product.
